SEQUENCE LISTING (41 sequences)

SEQ ID NO: 1 - protein, 21 aa, Flavivirus Zika
GDITWEKDAEVTGNSPRLDVA

SEQ ID NO: 2 - protein, 15 aa, Flavivirus Zika
GDITWEKDAEVTGNS

SEQ ID NO: 3 - protein, 15 aa, Flavivirus Zika
GDITWEKDAEITGNS

SEQ ID NO: 4 - protein, 15 aa, Flavivirus Zika
TWEKDAEVTGNSPRL

SEQ ID NO: 5 - protein, 15 aa, Flavivirus Zika
TWEKDAEITGNSPRL

SEQ ID NO: 6 - protein, 15 aa, Flavivirus Zika
KDAEVTGNSPRLDVA

SEQ ID NO: 7 - protein, 15 aa, Flavivirus Zika
KDAEITGNSPRLDVA

SEQ ID NO: 8 - protein, 21 aa, Flavivirus Zika
GDITWEKDAEITGNSPRLDVA

SEQ ID NO: 9 - protein, 15 aa, Flavivirus Zika
WEKDAEVTGNSPRLD

SEQ ID NO: 10 - protein, 9 aa, Flavivirus Zika
KDAEVTGNS

SEQ ID NO: 11 - protein, 15 aa, Artificial Sequence
  Peptide with SEQ ID NO.9 substituted in position 9 and 11
WEKDPEDTGNSPRLD

SEQ ID NO: 12 - protein, 15 aa, Artificial Sequence
  Peptide with SEQ ID NO.9 substituted in position 9
WEKDAEDTGNSPRLD

SEQ ID NO: 13 - protein, 15 aa, Artificial Sequence
  Peptide with SEQ ID NO.9 substituted in position 9, 11 and 15
WEKDPEDTGNKPRLD

SEQ ID NO: 14 - protein, 9 aa, Artificial Sequence
  Peptide with SEQ ID NO. 10 substituted in position 9 and 11
KDPEDTGNS

SEQ ID NO: 15 - protein, 9 aa, Artificial Sequence
  Peptide with SEQ ID NO. 10 substituted in position 9
KDAEDTGNS

SEQ ID NO: 16 - protein, 9 aa, Artificial Sequence
  Peptide with SEQ ID NO. 10 substituted in position 9, 11 and 15
KDPEDTGNK

SEQ ID NO: 17 - protein, 130 aa, Flavivirus Zika
SWPPSEVLTAVGLICALAGGFAKADIEMAGPMAAVGLLIVSYVVSGKSVDMYIERAGDITWEKDAEVTGNSPRLDVALDESGDFSLVEEDGPPMREIILKVVLMAICGMNPIAIPFAAGAWYVYVKTGKR

SEQ ID NO: 18 - protein, 21 aa, West Nile virus
ADITWESDAEITGSSERVDVR

SEQ ID NO: 19 - protein, 131 aa, West Nile virus
GWPATEVMTAVGLMFAIVGGLAELDIDSMAIPMTIAGLMFAAFVISGKSTDMWIERTADITWESDAEITGSSERVDVRLDDDGNFQLMNDPGAPWKIWMLRMACLAISAYTPWAILPSVIGFWITLQYTKR

SEQ ID NO: 20 - protein, 21 aa, Dengue virus type 1
AEVSWEEEAEHSGASHNILVE

SEQ ID NO: 21 - protein, 130 aa, Dengue virus type 1
SWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAISGVYPMSIPATLFVWYFWQKKKQR

SEQ ID NO: 22 - protein, 21 aa, Dengue virus type 2
ADVKWEDQAEISGSSPILSIT

SEQ ID NO: 23 - protein, 130 aa, Dengue virus type 2
SWPLNEAIMAVGMVSILASSLLKNDIPMTGPLVAGGPLTVCYVLTGRSADLELERAADVKWEDQAEISGSSPILSITISEDGSMSIKNEEEEQTLTILIRTGLLVISGLFPVSIPITAAAWYLWEVKKQR

SEQ ID NO: 24 - protein, 21 aa, Dengue virus type 3
PDVTWEEEAEQTGVSHNLMIT

SEQ ID NO: 25 - protein, 130 aa, Dengue virus type 3
SWPLNEGVMAVGLVSILASSLLRNDVPMAGPLVAGGLLIACYVITGTSADLTVEKAPDVTWEEEAEQTGVSHNLMITVDDDGTMRIKDDETENILTVLLKTALLIVSGIFPYSIPATLLVWHTWQKQTQR

SEQ ID NO: 26 - protein, 21 aa, Yellow fever virus
GEVSWEEEAEISGSSARYDVA

SEQ ID NO: 27 - protein, 130 aa, Yellow fever virus
SIPVNEALAAAGLVGVLAGLAFQEMENFLGPIAVGGLLMMLVSVAGRVDGLELKKLGEVSWEEEAEISGSSARYDVALSEQGEFKLLSEEKVPWDQVVMTSLALVGAALHPFALLLVLAGWLFHVRGARR

SEQ ID NO: 28 - protein, 21 aa, Dengue virus type 4
ANVQWDEMADITGSSPIIEVK

SEQ ID NO: 29 - protein, 130 aa, Dengue virus type 4
SWPLNEGIMAVGLVSLLGSALLKNDVPLAGPMVAGGLLLAAYVMSGSSADLSLEKAANVQWDEMADITGSSPIIEVKQDEDGSFSIRDVEETNMITLLVKLALITVSGLYPLAIPVTMTLWYMWQVKTQR

SEQ ID NO: 30 - protein, 21 aa, Tick-borne encephalitis virus
GCVEWYPELVNEGGEVSLRVR

SEQ ID NO: 31 - protein, 131 aa, Tick-borne encephalitis virus
SFSEPLTVVGVMLTLASGMMRHTSQEALCALAVASFLLLMLVLGTRKMQLVAEWSGCVEWYPELVNEGGEVSLRVRQDAMGNFHLTELEKEERMMAFWLIAGLAASAIHWSGILGVMGLWTLTEMLRSSRR

SEQ ID NO: 32 - protein, 21 aa, Japanese encephalitis virus
ATDMWLERAADISWEMDAAIT

SEQ ID NO: 33 - protein, 131 aa, Japanese encephalitis virus
GWPATEFLSAVGLMFAIVGGLAELDIESMSIPFMLAGLMAVSYVVSGKATDMWLERAADISWEMDAAITGSSRRLDVKLDDDGDFHLIDDPGVPWKVWVLRMSCIGLAALTPWAIVPAAFGYWLTLKTTKR

SEQ ID NO: 34 - protein, 15 aa, Artificial Sequence
  Peptide, derived from SEQ ID NO. 9, with K7 substitution, wherein X is any amino acid except K (lysine)
  misc_feature (3)..(3): Xaa can be any naturally occurring amino acid
WEXDAEVTGNSPRLD

SEQ ID NO: 35 - protein, 9 aa, Artificial Sequence
  Peptide, derived from SEQ ID NO. 10, with K7 substitution, wherein X is any amino acid except K (lysine)
  misc_feature (1)..(1): Xaa can be any naturally occurring amino acid
XDAEVTGNS

SEQ ID NO: 36 - protein, 15 aa, Artificial Sequence
  Peptide, derived from SEQ ID NO. 11, with K7 substitution, wherein X is any amino acid except K (lysine)
  misc_feature (3)..(3): Xaa can be any naturally occurring amino acid
WEXDPEDTGNSPRLD

SEQ ID NO: 37 - protein, 15 aa, Artificial Sequence
  Peptide, derived from SEQ ID NO. 12, with K7 substitution, wherein X is any amino acid except K (lysine)
  misc_feature (3)..(3): Xaa can be any naturally occurring amino acid
WEXDAEDTGNSPRLD

SEQ ID NO: 38 - protein, 15 aa, Artificial Sequence
  Peptide, derived from SEQ ID NO. 13, with K7 substitution, wherein X is any amino acid except K (lysine)
  misc_feature (3)..(3): Xaa can be any naturally occurring amino acid
WEXDPEDTGNKPRLD

SEQ ID NO: 39 - protein, 9 aa, Artificial Sequence
  Peptide, derived from SEQ ID NO. 14, with K7 substitution, wherein X is any amino acid except K (lysine)
  misc_feature (1)..(1): Xaa can be any naturally occurring amino acid
XDPEDTGNS

SEQ ID NO: 40 - protein, 9 aa, Artificial Sequence
  Peptide, derived from SEQ ID NO. 15, with K7 substitution, wherein X is any amino acid except K (lysine)
  misc_feature (1)..(1): Xaa can be any naturally occurring amino acid
XDAEDTGNS

SEQ ID NO: 41 - protein, 9 aa, Artificial Sequence
  Peptide, derived from SEQ ID NO. 16, with K7 substitution, wherein X is any amino acid except K (lysine)
  misc_feature (1)..(1): Xaa can be any naturally occurring amino acid
XDPEDTGNK

(19) United States
(12) Patent Application Publication
(10) Pub. No.: US 2020/0325183 A1
(43) Pub. Date: Oct. 15, 2020

(54) NS2B AS MARKER FOR ZIKA VIRUS INFECTIONS

(71) Applicant: PEPperPRINT GMBH, HEIDELBERG (DE)

(72) Inventors: THOMAS JAENISCH, DRIEDORF (DE); NICO FISCHER, OBERDERDINGEN (DE); FELIX LOEFFLER, POTSDAM (DE); RENATE SEKUL, SCHRIESHEIM (DE); VOLKER STADLER, OFTERSHEIM (DE); ERNESTO T. A. MARQUES, PITTSBURGH, PA (US)

(21) Appl. No.: 16/955,950

(22) PCT Filed: Dec. 21, 2018
(86) PCT No.: PCT/EP2018/086699

§ 371 Date: Jun. 19, 2020

(30) Foreign Application Priority Data
Dec. 21, 2017 (EP) 17209651.3

(51) Int. Cl. C07K 14/005; C07K 7/64; G01N 33/569

(52) U.S. Cl. CPC C07K 14/005; G01N 2333/185; G01N 33/56983; C07K 7/64

(57) ABSTRACT

The present invention relates to protein NS2b or fragment(s) thereof as biomarker or diagnostic marker for the diagnosis and/or prognosis of Zika virus infections. The present invention further relates to peptides and cyclic peptides, compositions and arrays and multimer compounds comprising them. The present invention further relates to a method for the diagnosis and/or prognosis of Zika virus infections, comprising the use of protein NS2b or fragment(s) thereof, or of the peptides, cyclic peptides, compositions and/or arrays in immunoassays. The present invention further relates to peptide-based compounds comprising at least one fragment of protein NS2b and at least one further component and to methods for the diagnosis and/or prognosis of Zika virus infections.

The present invention relates to protein NS2b or fragment(s) thereof as biomarker or diagnostic marker for the diagnosis and/or prognosis of Zika virus infections. The present invention further relates to peptides and cyclic peptides, compositions and arrays and multimer compounds comprising them. The present invention further relates to a method for the diagnosis and/or prognosis of Zika virus infections, comprising the use of protein NS2b or fragment(s) thereof, or of the peptides, cyclic peptides, compositions and/or arrays in immunoassays. The present invention further relates to peptide-based compounds comprising at least one fragment of protein NS2b and at least one further component and to methods for the diagnosis and/or prognosis of Zika virus infections.

### BACKGROUND OF THE INVENTION

Given the recent outbreak of Zika virus (ZIKV) in the Americas in 2015 and the association with microcephaly cases (Lover 2016; de Araújo et al., 2016) and neurological disorders, such as Guillain-Barre syndrome, ZIKV was recently placed as a public health emergency of international concern by the World Health Organization (WHO) (Lessler et al., 2016).

ZIKV is a flavivirus of the family Flaviviridae, transmitted by Aedes sp. Mosquitoes (Chakraborty et al., 2016), and closely related to dengue virus (DENV), West Nile virus (WNV) and Japanese encephalitis virus (JEV) and yellow fever virus (YFV) (Lazear et al., 2016).

Up to date there is no prophylactic treatment or vaccine available against ZIKV and disease control is limited to vector eradication strategies. The presumptive diagnosis is typically clinical, while confirmatory laboratory tests include classic virus isolation on cell culture and viral RNA detection by reverse transcriptase PCR (RT-PCR) in serum, saliva and/or urine samples within the first 5 or 6 days of infection (Dawes et al., 2016; Waggoner et al., 2016). ZIKV serology is usually performed by Enzyme-Linked Immunosorbent Assay (ELISA), using full-length viral proteins or linear peptides. However, due to high cross-reactivity of IgM and IgG antibodies between ZIKV and other related flavivirus, especially in endemic areas where co-circulation exists, confirmatory tests are necessary. Currently, confirmation testing is performed by plaque reduction neutralization test (PRNT), according to previous published protocols (Johnson et al., 2000; Martin et al., 2000; Kuno 2003; Maeda and Maeda, 2013).

To the present there are only very few commercially available serology kits for ZIKV antibody detection, moreover the specificity of these kits remains elusive given the high cross reactivity already reported among individuals who experienced consecutive flavivirus infections (PMID: 28094237 and PMID: 27982355). Based on that, PRNT remains as the “gold standard” for anti-flavivirus differentiation (Kuno 2003). PRNT, however, is a high cost technique that requires highly specialized laboratories and special regulation due to live virus manipulation. Although new protocols using recombinant viruses (Johnson et al., 2009) or reporter virus particles (Maeda and Maeda, 2013) have been developed, these are still not available for ZIKV routine diagnosis (Musso and Gubler, 216).

Thus, there is an urgent need for a low-cost unequivocal serological diagnostic method for ZIKV able to overcome the high cross reactivity with other flaviviruses, such as DENV, and yellow fever (especially on vaccinated individuals) (Stettler et al., 2016).

There is a need in the art of improved means and methods for diagnosing Zika virus infections.

### SUMMARY OF THE INVENTION

According to the present invention this object is solved by protein NS2b as biomarker or diagnostic marker for use in a method for the diagnosis and/or prognosis of Zika virus infections.

According to the present invention this object is solved by at least one fragment of protein NS2b as biomarker or diagnostic marker for use in a method for the diagnosis and/or prognosis of Zika virus infections.

According to the present invention this object is solved by at least one mutated fragment of protein NS2b as biomarker or diagnostic marker for use in a method for the diagnosis and/or prognosis of Zika virus infections.

According to the present invention this object is solved by a peptide comprising an amino acid sequence selected from SEQ ID Nos. 1 to 10 having one, two, three or four amino acid substitution(s) in position 9, 11, 15 and/or 17 in reference to the amino acid sequence of SEQ ID NO. 1.

According to the present invention this object is solved by a cyclic peptide comprising
an amino acid sequence selected from SEQ ID Nos. 1 to 10, or
an amino acid sequence selected from SEQ ID Nos. 1 to 10 having one, two, three or four amino acid substitution(s) in position 9, 11, 15 and/or 17 in reference to the amino acid sequence of SEQ ID NO. 1.

According to the present invention this object is solved by a composition or an array comprising
(a) at least one peptide comprising
   an amino acid sequence selected from SEQ ID NOs. 1 to 10, or
   an amino acid sequence having at least about 50% sequence identity to an amino acid sequence of SEQ ID NOs. 1 to 10, preferably more than about 60%, more preferably more than about 70 or 80 or 90% sequence identity, or
(b) at least one peptide comprising
   an amino acid sequence selected from SEQ ID Nos. 1 to 10 having one, two, three or four amino acid substitution(s) in position 9, 11, 15 and/or 17 in reference to the amino acid sequence of SEQ ID NO. 1,
      and/or
(c) at least one cyclic peptide according to the present invention.

According to the present invention this object is solved by a multimer compound comprising at least two of component(s) (a) to (d):
(a) a peptide comprising
   an amino acid sequence selected from SEQ ID NOs. 1 to 10, or
   an amino acid sequence having at least about 50% sequence identity to an amino acid sequence of SEQ ID NOs. 1 to 10, preferably more than about 60%, more preferably more than about 70 or 80 or 90% sequence identity, or
(b) a peptide of the present invention,
(c) a cyclic peptide of the present invention, and/or
(d) a NS2b fragment, as defined herein,
   wherein at least two of component(s) (a) to (d) are covalently coupled to each other or are connected via a linear or cyclic scaffold.

According to the present invention this object is solved by providing the peptide or the cyclic peptide according to the present invention or the composition or array of the present invention or the multimer compound of the present invention as biomarker or diagnostic marker for Zika virus infections.

According to the present invention this object is solved by peptide or the cyclic peptide according to the present invention or the composition or array of the present invention or the multimer compound of the present invention for use in a method of the diagnosis and/or prognosis of Zika virus infections.

According to the present invention this object is solved by a peptide-based compound comprising
(i) at least one peptide comprising
   an amino acid sequence selected from SEQ ID NOs. 1 to 10, or
   an amino acid sequence having at least about 50% sequence identity to an amino acid sequence of SEQ ID NOs. 1 to 10, preferably more than about 60%, more preferably more than about 70 or 80 or 90% sequence identity, or
   an amino acid sequence selected from SEQ ID Nos. 1 to 10 having one, two, three or four amino acid substitution(s) in position 9, 11, 15 and/or 17 in reference to the amino acid sequence of SEQ ID NO. 1,
   and/or at least one cyclic peptide according to the present invention,
(ii) at least one further component, preferably covalently coupled to the peptide and/or cyclic peptide (i).

According to the present invention this object is solved by providing a peptide-based compound of the present invention for use in a method of the diagnosis and/or prognosis of Zika virus infections.

According to the present invention this object is solved by an in vitro method for the diagnosis and/or prognosis of Zika virus infections, comprising the step of

(a) providing a sample of a patient to be tested,
(b) providing
(1) protein NS2b or at least one fragment thereof, as defined herein;
(2) at least one peptide-based compound of the present invention;
(3) at least one peptide of the present invention;
(4) at least one cyclic peptide of the present invention;
(5) a composition or an array of the present invention;
and/or
(6) a multimer compound of the present invention
   (c) performing an immunoassay, comprising detecting an anti-Zika antibody response in said patient sample.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific tennis used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “5 to 100” should be interpreted to include not only the explicitly recited values of 5 to 100, but also include individual values and sub-ranges within the indicated range. Thus, included in this numerical range are individual values such as 5, 6, 7, 8, 9, 10, 11, 12, 13 . . . 97, 98, 99, 100 and sub-ranges such as from 10 to 40, from 12 to 17 and 41 to 50, etc. This same principle applies to ranges reciting only one numerical value, such as “at least 8”. Furthermore, such an interpretation should apply regardless of the breadth of the range or the characteristics being described. Also it is to be understood that ranges may differ depending on the institute/facility where the measurements are being performed, methodology of measurement, type of tissue, and technique of tissue collection.

Biomarker for Zika Virus

As discussed above, the present invention provides protein NS2b as biomarker or diagnostic marker for Zika virus infections.

In particular, the present invention provides protein NS2b as biomarker or diagnostic marker for use in a method for the diagnosis and/or prognosis of Zika virus infections.

Furthermore, the present invention provides at least one fragment of protein NS2b as biomarker or diagnostic marker for use in a method for the diagnosis and/or prognosis of Zika virus infections.

Furthermore, the present invention provides at least one mutated fragment of protein NS2b as biomarker or diagnostic marker for use in a method for the diagnosis and/or prognosis of Zika virus infections.

The ZIKV positive-sense RNA genome comprises a single ORF (Open Reading Frame) encoding an unique polyprotein that is cleaved into three structural proteins (Capsid (C), pre-membrane (prM) and envelope (E), which form the virus particle, and seven non-structural (NS) proteins (NS1, NS2A, NS2B, NS3, NS4A, NS4B and NS5), which perform essential functions in genome replication, polyprotein cleavage and modulation of cellular processes to viral advantage (Kuno et al., 2007; Brown et al., 2016).

Several of the non-structural proteins function as enzymes for the virus. Among the NS proteins, the NS1 glycoprotein is a multifunctional virulence factor (Muller and Young, 2013; Watterson et al., 2016). Furthermore there is the serine protease NS3, whose function is to cleave the virus polyprotein at proper sites, and is required for ZIKV replication. NS3 requires a cofactor, NS2b. The crystal structure of the NS2b-NS3 complex was recently resolved by Zhang et al. (2016).

In a preferred embodiment, the least one fragment of NS2b is an amino acid sequence of the ZIKV polyproteome of positions 1429-1449 of Zika Uganda Strain MR766 NIID (SEQ ID NO. 1).

Said amino acid sequence of SEQ ID NO. 1 is
GDITWEKDAEVTGNSPRLDVA

Said amino acid sequence of the ZIKV polyproteome was identified by the inventors as a major epitope. Said amino acid sequence refers, for example, to
positions 1429-1449 of Zika Uganda Strain MR766_NIID; or
positions 1425-1445 of Zika Uganda Strain MR766.
See Table 1 below.

It is identical in the Zika strains (except for one V→I exchange, namely the central valine in position 11), such as the 15 Zika strains used for identification. See Table 1 below.

When compared to the corresponding regions of NS2b of other flaviviruses (as identified from Uniprot), it can be seen that the epitope of Zika virus differs, such as, among others, in position 7 (lysine), and is highly suitable for a specific diagnosis of Zika infections.

Zika virus
SEQ ID NO. 1
GDITWEKDAEVTGNSPRLDVA
West Nile Virus
SEQ ID NO. 18
ADITWESDAEITGSSERVDVR
DENV-1
SEQ ID NO. 20
AEVSWEEEAEHSGASHNILVE
DENV-2
SEQ ID NO. 22
ADVKWEDQAEISGSSPILSIT
DENV-3
SEQ ID NO. 24
PDVTWEEEAEQTGVSHNLMIT
Yellow fever virus
SEQ ID NO. 26
GEVSWEEEAEISGSSARYDVA
DENV-4
SEQ ID NO. 28
ANVQWDEMADITGSSPIIEVK
TBEV
SEQ ID NO. 30
GCVEWYPELVNEGGEVSLRVR
JEV
SEQ ID NO. 32
ATDMWLERAADISWEMDAAIT.

The amino acid sequences of the full length NS2b of the flaviviruses are (as identified from Uniprot)

Zika virus
Uniprot Accession No. Q32ZE1; Zika Strain MR766
1369-1498 NS2b
SEQ ID NO. 17
SWPPSEVLTAVGLICALAGGFAKADIEMAGPMAAVGLLIVSYVVSGKSVDM
YIERAGDITWEKDAEVTGNSPRLDVALDESGDFSLVEEDGPPMREIILKVV
LMAICGMNPIAIPFAAGAWYVYVKTGKR
West Nile virus (WNV)
Uniprot Accession No. P06935
1371-1501 NS2b
SEQ ID NO. 19
GWPATEVMTAVGLMFAIVGGLAELDIDSMAIPMTIAGLMFAAFVISGKSTD
MWIERTADITWESDAEITGSSERVDVRLDDDGNFQLMNDPGAPWKIWMLRM
ACLAISAYTPWAILPSVIGFWITLQYTKR
Dengue virus type 1 (DENV-1)
Uniprot Accession No. P17763
1346-1475 NS2b
SEQ ID NO. 21
SWPLNEGIMAVGIVSILLSSLLKNDVPLAGPLIAGGMLIACYVISGSSADL
SLEKAAEVSWEEEAEHSGASHNILVEVQDDGTMKIKDEERDDTLTILLKAT
LLAISGVYPMSIPATLFVWYFWQKKKQR
Dengue virus type 2 (DENV-2)
Uniprot Accession No. P29990
1346-1475 NS2b
SEQ ID NO. 23
SWPLNEAIMAVGMVSILASSLLKNDIPMTGPLVAGGPLTVCYVLTGRSADL
ELERAADVKWEDQAEISGSSPILSITISEDGSMSIKNEEEEQTLTILIRTG
LLVISGLFPVSIPITAAAWYLWEVKKQR
Dengue virus type 3 (DENV-3)
Uniprot Accession No. Q6YMS4
1344-4473 NS2b
SEQ ID NO. 25
SWPLNEGVMAVGLVSILASSLLRNDVPMAGPLVAGGLLIACYVITGTSADL
TVEKAPDVTWEEEAEQTGVSHNLMITVDDDGTMRIKDDETENILTVLLKTA
LLIVSGIFPYSIPATLLVWHTWQKQTQR
Yellow fever virus (YFV)
Uniprot Accession No. P03314
1355-1484 NS2b
SEQ ID NO. 27
SIPVNEALAAAGLVGVLAGLAFQEMENFLGPIAVGGLLMMLVSVAGRVDGL
ELKKLGEVSWEEEAEISGSSARYDVALSEQGEFKLLSEEKVPWDQVVMTSL
ALVGAALHPFALLLVLAGWLFHVRGARR
Dengue virus type 3 (DENV-4)
Uniprot Accession No. Q5UCB8
1345-1474 NS2b
SEQ ID NO. 29
SWPLNEGIMAVGLVSLLGSALLKNDVPLAGPMVAGGLLLAAYVMSGSSADL
SLEKAANVQWDEMADITGSSPIIEVKQDEDGSFSIRDVEETNMITLLVKLA
LITVSGLYPLAIPVTMTLWYMWQVKTQR
Tick-borne encephalitis virus (TBEV)
Uniprot Accession No. P14336
1359-1489 NS2b
SEQ ID NO. 31
SFSEPLTVVGVMLTLASGMMRHTSQEALCALAVASFLLLMLVLGTRKMQLV
AEWSGCVEWYPELVNEGGEVSLRVRQDAMGNFHLTELEKEERMMAFWLIAG
LAASAIHWSGILGVMGLWTLTEMLRSSRR
Japanese encephalitis virus (JEV)
Uniprot Accession No. P27395
1374-1504 NS2b
SEQ ID NO. 33
GWPATEFLSAVGLMFAIVGGLAELDIESMSIPFMLAGLMAVSYVVSGKATD
MWLERAADISWEMDAAITGSSRRLDVKLDDDGDFHLIDDPGVPWKVWVLRM
SCIGLAALTPWAIVPAAFGYWLTLKTTKR

Preferably, the fragment is a peptide having a length of at least 5 amino acids, or at least 9 amino acids,

such as 5 to 130 amino acids, or 5 to 100 amino acids, or 5 to 50 amino acids, or 9 to 50 amino acids, e.g. about 15 to 21 amino acids or about 9 to 15 amino acids or about 9 to 21 amino acids.

Other lengths will be determined by the skilled artisan depending on the use.

In a preferred embodiment, the at least one fragment comprises or is or consists of a peptide having
an amino acid sequence selected from SEQ ID Nos. 1 to 10 or combinations thereof, or
an amino acid sequence having at least about 50% sequence identity to an amino acid sequence of SEQ ID NOs. 1 to 10, preferably more than about 60%, more preferably more than about 70 or 80 or 90% sequence identity.

SEQ ID NO. 2
GDITWEKDAEVTGNS;
SEQ ID NO. 3
GDITWEKDAEITGNS;
SEQ ID NO. 4
TWEKDAEVTGNSPRL;
SEQ ID NO. 5
TWEKDAEITGNSPRL;
SEQ ID NO. 6
KDAEVTGNSPRLDVA;
SEQ ID NO. 7
KDAEITGNSPRLDVA;
SEQ ID NO. 1
GDITWEKDAEVTGNSPRLDVA;
SEQ ID NO. 8
GDITWEKDAEITGNSPRLDVA;
SEQ ID NO. 9
WEKDAEVTGNSPRLD;
SEQ ID NO. 10
KDAEVTGNS.

In one embodiment, the NS2b fragments can comprise non-natural amino acids, D-amino acids, modified amino acids and/or amino acid derivatives, such as β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, N-methyl amino acids.

Examples are ornithine, citrulline, norleucine, acetyllysine, phosphotyrosine.

In a preferred embodiment, the NS2b fragment does not comprise an amino acid substitution in position K7.

An amino acid substitution in position K7 will result in a peptide/NS2b fragment which will no longer detect Zika virus infections, as described herein below.

In one embodiment, the NS2b fragment comprises or is or consists of a peptide having
an amino acid sequence selected from SEQ ID Nos. 1 to 10, or
an amino acid sequence selected from SEQ ID Nos. 1 to 10 having one, two, three or four amino acid substitution(s) in position 9, 11, 15 and/or 17 in reference to the amino acid sequence of SEQ ID NO. 1.

Positions 9, 11, 15 and 17 in amino acid sequence of SEQ ID NO. 1: (underlined)

SEQ ID NO. 1
GDITWEKDAEVTGNSPRLDVA

The amino acid substitution(s) are preferably
A9P, A9E, A9M, A9S, A9T, A9K;
V11D, V11E, V11T, V11A, V11N, V11S, V11M, V11L or I11D, I11E, I11T, I11A, I11N, I11S, I11M, I11L;
S15D, S15K, S15M, S15A, S15R, S15N; and/or
R17D, R17E, R17T
   more preferably
A9P, A9E,
V11D, MD, V11E, I11E,
S15D, S15K, and/or
R17D, R17E.

The amino acid substitution is not any substitution in position K7.

For example, the NS2b fragment comprises or is or consists of a peptide having an amino acid sequence selected from SEQ ID Nos. 11 to 16.

SEQ ID NO. 11
WEKDPEDTGNSPRLD
SEQ ID NO. 12
WEKDAEDTGNSPRLD
SEQ ID NO. 13
WEKDPEDTGNKPRLD
SEQ ID NO. 14
KDPEDTGNS
SEQ ID NO. 15
KDAEDTGNS
SEQ ID NO. 16
KDPEDTGNK

In one embodiment, the NS2b fragment comprises or is or consists of a cyclic peptide having
an amino acid sequence selected from SEQ ID Nos. 1 to 10, or
an amino acid sequence selected from SEQ ID Nos. 1 to 10 having one, two, three or four amino acid substitution(s) in position 9, 11, 15 and/or 17 in reference to the amino acid sequence of SEQ ID NO. 1.

The cyclization is preferably via
thioether formation, wherein the peptide comprises an additional cysteine residue at or near the C-terminus, or
head-to-tail cyclization, or
disulfide formation via two additional cysteine residues at or near the C- and the N-terminus, or
lactam formation via a C- or N-terminal located additional basic amino acid, such as lysine or ornithine, and a C- or N-terminal located acidic amino acid, such as glutamic acid or aspartic acid, or
triazole formation via a C- or N-terminal located additional amino acid with an alkyne side chain and a C- or N-terminal located additional amino acid with an azide side chain.

More preferably, the cyclization is via thioether formation or head-to-tail cyclization.

Preferably,
(i) one fragment is used which comprises more than one of said peptides, or
(ii) more than one fragment is used each comprising or consisting of one peptide.

In one embodiment (i), wherein one fragment is used which comprises more than one of said peptides, the more than one of said peptides are covalently coupled to each other and/or are coupled to a linear or cyclic scaffold. The skilled artisan is able to generate such coupled peptides and/or scaffolds and knows appropriate linear and cyclic scaffolds.

In one embodiment, said NS2b protein or said at least one fragment comprises at least one further component(s).

Said at least one further component(s) is/are preferably selected from
label(s),
   such as fluorescent label(s),
tag(s),
   preferably for immobilization, such as biotin, 6×His, alkyn, azid, thiol,
linker or anchoring group(s)
   preferably for attachment of the label(s), and/or tag(s),
   such as PEG, cysteine, poly-GS-linker, e.g. GSGSG, or βAla-βAla-linker, or combinations thereof.

Said at least one further component(s) is/are preferably covalently coupled to said NS2b protein or said at least one fragment thereof
via a linker,
via an amino acid side chain, and/or
to the N- and/or C-terminus.

Said method is an in vitro, ex vivo or in vivo method.

In a preferred embodiment, the use of NS2b protein or at least one fragment thereof comprises the use in immunoassays.

Immunoassays are known to the skilled artisan and comprise, for example,
enzyme-linked immunosorbent assay (ELISA),
bead-based immunoassay, such as Luminex,
plaque reduction neutralization test (PRNT),
lateral flow or strip tests,
encoded microparticle-based immunoassay,
magnetic bead-based immunoassay.

Preferably, detecting an anti-Zika antibody response in a sample of a patient is comprised.

Said patient sample is preferably blood. More preferably it is selected from serum, plasma and whole blood.

Substituted Peptides, Cyclic Peptides, Compositions and Arrays and Multimer Compounds and their Uses

As discussed above, the present invention provides a peptide comprising an amino acid sequence selected from SEQ ID Nos. 1 to 10 having one, two, three or four amino acid substitution(s) in position 9, 11, 15 and/or 17 in reference to the amino acid sequence of SEQ ID NO. 1.

Positions 9, 11, 15 and 17 in amino acid sequence of SEQ ID NO. 1: (underlined), as described above:

SEQ ID NO. 1
GDITWEKDAEVTGNSPRLDVA

The amino acid substitution(s) are preferably
A9P, A9E, A9M, A9S, A9T, A9K;
V11D, V11E, V11T, V11A, V11N, V11S, V11M, V11L or I11D, I11E, I11T, I11A, I11N, I11S, I11M, I11L;
S15D, S 15K, S15M, S15A, S15R, S15N; and/or
R17D, R17E, R17T
   more preferably
A9P, A9E,
V11D, MD, V11E, I11E,
S15D, S15K, and/or
R17D, R17E.

The amino acid substitution is not any substitution in position K7.

For example, the peptide comprises or is or consists of an amino acid sequence selected from SEQ ID Nos. 11 to 16.

SEQ ID NO. 11
WEKDPEDTGNSPRLD
SEQ ID NO. 12
WEKDAEDTGNSPRLD
SEQ ID NO. 13
WEKDPEDTGNKPRLD
SEQ ID NO. 14
KDPEDTGNS
SEQ ID NO. 15
KDAEDTGNS
SEQ ID NO. 16
KDPEDTGNK

In a preferred embodiment, the peptide of the present invention does not comprise an amino acid substitution in position K7.

An amino acid substitution in position K7 will result in a peptide/NS2b fragment which will no longer detect Zika virus infections, as described herein below.

As discussed above, the present invention provides a cyclic peptide comprising
an amino acid sequence selected from SEQ ID Nos. 1 to 10, or
an amino acid sequence selected from SEQ ID Nos. 1 to 10 having one, two, three or four amino acid substitution(s) in position 9, 11, 15 and/or 17 in reference to the amino acid sequence of SEQ ID NO. 1.

In a preferred embodiment, the cyclization is via
thioether formation, wherein the peptide comprises an additional cysteine residue at or near the C-terminus,
Or
head-to-tail cyclization.

The amino acid substitution(s) are preferably, as described above:
A9P, A9E, A9M, A9S, A9T, A9K;
V11D, V11E, V11T, V11A, V11N, V11S, V11M, V11L or I11D, I11E, I11T, I11A, I11N, I11S, I11M, I11L;
S15D, S15K, S15M, S15A, S15R, S15N; and/or
R17D, R17E, R17T
   more preferably
A9P, A9E,
V11D, MD, V11E, I11E,
S15D, S15K, and/or
R17D, R17E.

The amino acid substitution is not any substitution in position K7.

Preferred examples for cyclic peptides according to the present invention are:

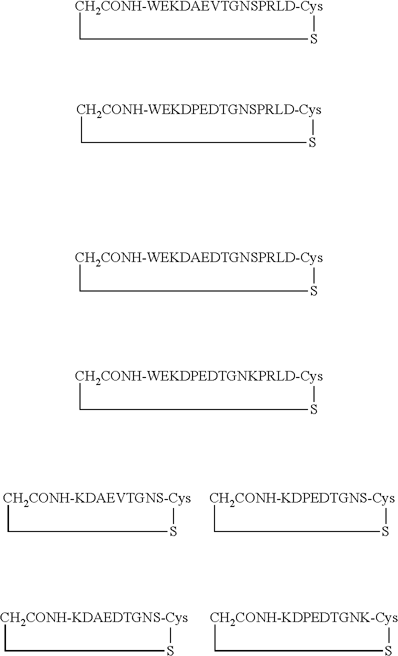

The cyclic peptides shown comprise an additional cysteine residue at the C-terminus. The cyclization is via thioether formation with the N-terminus, which carried a bromoacetyl group as chemical handle for the thioether formation.

Further possibilities for cyclization are known to the skilled artisan. Cyclization is e.g. also possible via

disulfide formation via two additional cysteine residues at or near the C- and the N-terminus, or
lactam formation via a C- or N-terminal located additional basic amino acid, such as lysine or ornithine, and a C- or N-terminal located acidic amino acid, such as glutamic acid or aspartic acid, or
triazole formation via a C- or N-terminal located additional amino acid with an alkyne side chain and a C- or N-terminal located additional amino acid with an azide side chain.

Preferably, the peptide(s) or the cyclic peptide(s) of the present invention have a length of at least 5 amino acids, or at least 9 amino acids,

such as 5 to 130 amino acids, or 5 to 100 amino acids, or 5 to 50 amino acids, or 9 to 50 amino acids, e.g. about 15 to 21 amino acids or about 9 to 15 amino acids or about 9 to 21 amino acids.

Other lengths will be determined by the skilled artisan depending on the use.

Preferably, the peptide or the cyclic peptide comprise at least one further component(s), which is/are preferably selected from
label(s),
   such as fluorescent label(s),
tag(s),
   preferably for immobilization, such as biotin, 6×His, alkyn, azid, thiol,
linker or anchoring group(s)
   preferably for attachment of the label(s), and/or tag(s),
   such as PEG, cysteine, poly-GS-linker, e.g. GSGSG,
or combinations thereof,
   wherein said at least one further component(s) is/are covalently coupled to said peptide via a linker, an amino acid side chain, and/or to the N- and/or C-terminus.

In one embodiment, the NS2b fragments can comprise non-natural amino acids, D-amino acids, modified amino acids and/or amino acid derivatives, such as β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, N-methyl amino acids.

Examples are ornithine, citrulline, norleucine, acetyllysine, phosphotyrosine.

As discussed above, the present invention provides a composition or an array comprising
(a) at least one peptide comprising
   an amino acid sequence selected from SEQ ID NOs. 1 to 10, or
   an amino acid sequence having at least about 50% sequence identity to an amino acid sequence of SEQ ID NOs. 1 to 10, preferably more than about 60%, more preferably more than about 70 or 80 or 90% sequence identity, or
(b) at least one peptide comprising
   an amino acid sequence selected from SEQ ID Nos. 1 to 10 having one, two, three or four amino acid substitution(s) in position 9, 11, 15 and/or 17 in reference to the amino acid sequence of SEQ ID NO. 1,
      and/or
(c) at least one cyclic peptide according to the present invention.

The preferred amino acid substitution(s) are as discussed above.

An “array” according to the present invention is preferably a peptide array or peptide microarray. Peptide (micro)arrays are known in the art. A peptide (micro)array is a number of peptides displayed or assembled on a solid surface, usually a glass or plastic chip or beads, silicone wafer.

A preferred surface for an array according to the invention is glass (chip).

An exemplary array is a bead-based array.

A composition according to the present invention is preferably a mixture of several of the peptides and/or cyclic peptides. A composition can comprise excipient(s) and/or carrier, such as pharmaceutically active excipient(s) and/or carrier.

As discussed above, the present invention provides a multimer compound comprising at least two of component(s) (a) to (d):
(a) a peptide comprising
   an amino acid sequence selected from SEQ ID NOs. 1 to 10, or
   an amino acid sequence having at least about 50% sequence identity to an amino acid sequence of SEQ ID NOs. 1 to 10, preferably more than about 60%, more preferably more than about 70 or 80 or 90% sequence identity, or
(b) a peptide of the present invention,
(c) a cyclic peptide of the present invention,
   and/or
(d) a NS2b fragment, as defined herein.

The peptide of the present invention (b) is as defined herein above: a peptide comprising an amino acid sequence selected from SEQ ID Nos. 1 to 10 having one, two, three or four amino acid substitution(s) in position 9, 11, 15 and/or 17 in reference to the amino acid sequence of SEQ ID NO. 1. The preferred amino acid substitution(s) are as discussed above.

The at least two of component(s) (a) to (d) are covalently coupled to each other or are connected via a linear or cyclic scaffold.

The skilled artisan knows appropriate linear and cyclic scaffolds and is able to couple the component(s) with each other and/or to the scaffolds.

The at least two of component(s) (a) to (d) can be e.g. at least two components (a), or at least two components (b), or at least two components (c) and/or at least two components (d). The at least two of component(s) (a) to (d) can be e.g. three components (a), or three components (b), or five components (c) and/or four components (d).

The at least two of component(s) (a) to (d) can be e.g. component (a)+(b), or (a)+(b)+(c) etc.

As discussed above, the present invention provides the peptide or the cyclic peptide according to the present invention as biomarker or diagnostic marker for Zika virus infections.

As discussed above, the present invention provides the composition or array of the present invention as biomarker or diagnostic marker for Zika virus infections.

As discussed above, the present invention provides the multimer compound of the present invention as biomarker or diagnostic marker for Zika virus infections.

As discussed above, the present invention provides the peptide or the cyclic peptide according to the present invention for use in a method of the diagnosis and/or prognosis of Zika virus infections, wherein said method is an in vitro, ex vivo or in vivo method, as described herein.

As discussed above, the present invention provides the composition or array of the present invention for use in a method of the diagnosis and/or prognosis of Zika virus infections, wherein said method is an in vitro, ex vivo or in vivo method, as described herein.

As discussed above, the present invention provides the multimer compound of the present invention for use in a method of the diagnosis and/or prognosis of Zika virus infections, wherein said method is an in vitro, ex vivo or in vivo method, as described herein.

Biomarker Compounds and their Uses

As discussed above, the present invention provides a peptide-based compound comprising
(i) at least one peptide comprising an amino acid sequence selected from SEQ ID NOs. 1 to 10

SEQ ID NO. 2
GDITWEKDAEVTGNS;
SEQ ID NO. 3
GDITWEKDAEITGNS;
SEQ ID NO. 4
TWEKDAEVTGNSPRL;
SEQ ID NO. 5
TWEKDAEITGNSPRL;
SEQ ID NO. 6
KDAEVTGNSPRLDVA;
SEQ ID NO. 7
KDAEITGNSPRLDVA;
SEQ ID NO. 1
GDITWEKDAEVTGNSPRLDVA;
SEQ ID NO. 8
GDITWEKDAEITGNSPRLDVA;
SEQ ID NO. 9
WEKDAEVTGNSPRLD;
SEQ ID NO. 10
KDAEVTGNS.

or an amino acid sequence having at least about 50% sequence identity to an amino acid sequence of SEQ ID NOs. 1 to 10, preferably more than about 60%, more preferably more than about 70 or 80 or 90% sequence identity,
and/or
an amino acid sequence selected from SEQ ID Nos. 1 to 10 having one, two, three or four amino acid substitution(s) in position 9, 11, 15 and/or 17 in reference to the amino acid sequence of SEQ ID NO. 1,
and/or
at least one cyclic peptide according to the present invention,
and
(ii) at least one further component, preferably covalently coupled to the peptide and/or cyclic peptide (i).

Preferably, the peptide(s) or the cyclic peptide(s) of the present invention have a length of at least 5 amino acids, or at least 9 amino acids,

such as 5 to 130 amino acids, or 5 to 100 amino acids, or 5 to 50 amino acids, or 9 to 50 amino acids, e.g. about 15 to 21 amino acids or about 9 to 15 amino acids or about 9 to 21 amino acids.

In one embodiment, the NS2b fragments can comprise non-natural amino acids, D-amino acids, modified amino acids and/or amino acid derivatives, such as β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, N-methyl amino acids.

Examples are ornithine, citrulline, norleucine, acetyllysine, phosphotyrosine.

Preferably, the at least one further component (ii) is selected from
label(s), such as fluorescent label(s),
tag(s), preferably for immobilization, such as biotin, 6×His, alkyn, azid, thiol,
linker or anchoring group(s) preferably for attachment of the label(s), and/or tag(s), such as PEG, cysteine, poly-GS-linker, e.g. GSGSG, or combinations thereof.

Said at least one further component(s) (ii) is/are preferably covalently coupled to said NS2b protein or said at least one fragment thereof via a linker, an amino acid side chain, and/or to the N- and/or C-terminus.

As discussed above, the present invention provides the peptide-based compound for use in a method of the diagnosis and/or prognosis of Zika virus infections.

Said method is an in vitro, ex vivo or in vivo method.

Preferably, the method comprises the use of immunoassays.

Immunoassays are known to the skilled artisan and comprise, for example,
enzyme-linked immunosorbent assay (ELISA),
bead-based immunoassay, such as Luminex,
plaque reduction neutralization test (PRNT),
lateral flow or strip tests
encoded microparticle-based immunoassay,
magnetic bead-based immunoassay.

Preferably, the method comprises detecting an anti-Zika antibody response in a sample of a patient.

Said patient sample is preferably blood. More preferably it is selected from serum, plasma and whole blood.

Methods for Diagnosis and/or Prognosis

As discussed above, the present invention provides an in vitro or ex vivo method for the diagnosis and/or prognosis of Zika virus infections.

Said method comprises the steps of

(a) providing a sample of a patient to be tested,

(b) providing
(1) protein NS2b or at least one fragment thereof, as defined herein;
(2) at least one peptide-based compound of the present invention;
(3) at least one peptide of the present invention;
(4) at least one cyclic peptide of the present invention;
(5) a composition or an array of the present invention;
   and/or
(6) a multimer compound of the present invention;

(c) performing an immunoassay, comprising detecting an anti-Zika antibody response in said patient sample.

Immunoassays are known to the skilled artisan and comprise, for example,
enzyme-linked immunosorbent assay (ELISA),
bead-based immunoassay, such as Luminex,
plaque reduction neutralization test (PRNT),
lateral flow or strip tests
encoded microparticle-based immunoassay,
magnetic bead-based immunoassay.

Said patient sample is preferably blood. More preferably it is selected from serum, plasma and whole blood.

In a preferred embodiment, a negative control is used, which preferably excludes a Zika virus infection.

A preferred negative control is a peptide, a cyclic peptide or a peptide-based compound according to the present invention comprising an amino acid substitution in position K7 in reference to the amino acid sequence of SEQ ID NO. 1.

Examples for such negative controls are peptide(s), cyclic peptide(s) and/or peptide-based compound(s) comprising or consisting of an amino acid sequence selected from

SEQ ID NO. 34
WEXDAEVTGNSPRLD
SEQ ID NO. 35
XDAEVTGNS
SEQ ID NO. 36
WEXDPEDTGNSPRLD
SEQ ID NO. 37
WEXDAEDTGNSPRLD
SEQ ID NO. 38
WEXDPEDTGNKPRLD
SEQ ID NO. 39
XDPEDTGNS
SEQ ID NO. 40
XDAEDTGNS
SEQ ID NO. 41
XDPEDTGNK

wherein, in each sequence, X is any amino acid except K (lysine).

### Further Description of Preferred Embodiments

In a first study, the inventors characterized the antibody profiles of Zika patients in a systematic manner. Therefore, they translated the proteomes of 15 different ZIKV strains (taken from the NCBI database, see Table 1 below) in the form of overlapping peptides as high-density peptide arrays and used 84 different patient and control sera to screen several thousands of potential antigens for pathogen induced antibodies.

TABLE 1
ZIKV strains contained in the NCBI database

| | | Proteome | |
|---|---|---|---|
| | | length | NCBI |
| | | (in amino | GeneBank |
| # | Strain ID | acids) | Accession No. |
| 1 | ZikaUgandaStrainMR766-NIID | 3423 | LC002520.1 |
| 2 | ZikaUgandaStrainMR766 | 3419 | Q32ZE1 |
| 3 | ZikaChinaStrainVE_Ganxian | 3423 | KU744693.1 |
| 4 | ZikaPhilStrainCPC-0740 | 3423 | KU681082.3 |
| 5 | ZikaThaiStrainSV0127-14 | 3423 | KU681081.3 |
| 6 | ZikaChinaStrainZJ03 | 3423 | KU761560.1 or |
| | | | KU820899.2 |
| 7 | ZikaHaitiStrain1225/2014 | 3423 | KU509998.3 |
| 8 | ZikaBrazilStrainSPH2015 | 3423 | KU321639.1 |
| 9 | ZikaBrazilStrainNatalRGN | 3423 | KU527068.1 |
| 10 | ZikaDomRepStrainPD2 | 3423 | KU853013.1 |
| 11 | ZikaDomRepStrainPD1 | 3423 | KU853012.1 |
| 12 | ZikaChinaStrainGD01 | 3423 | KU740184.2 |
| 13 | ZikaBrazilStrainZKV2015 | 3423 | KU497555.1 |
| 14 | ZikaBrazilStrainSSABR1 | 3423 | KU707826.1 |
| 15 | ZikaPuertoRicoStrainPRVABC59 | 3423 | KU501215.1 |

All strains carry the major epitope in polyproteome position 1429-1449, except strain #2, where it is located at position 1425-1445.

In addition, the inventors investigated the time-dependent antibody variation in patients (acute vs. convalescent stage). They screened patient sera from their collaboration partner in Brazil with different peptide arrays, containing the whole proteome of the ZIKV (first screening round). Analyses showed specific IgG responses towards NS2b, especially in convalescent samples. Almost no signals could be observed in control samples. Additional samples have been recently screened for specific reactivity towards three proteins (NS1, NS2a, NS2b).

In a first screening round of the first study, arrays with whole proteome content were used. The IgG antibody reactivity of Zika patients of different groups was screened against the whole ZIKV proteome (see FIG. 2, only protein NS2b shown). The most prominent interaction was observed with the protein NS2b.

In a second screening round of the first study, specific arrays with NS1. NS2a. NS2b content were used. The IgG antibody reactivity showed clear signals in the NS2b protein. A major epitope was identified. See FIG. 3. The major epitope is GDITWEKDAEVTGNSPRLDVA [SEQ ID NO. 1] or GDITWEKDAEITGNSPRLDVA (V->I) [SEQ ID NO. 8], maximum length 21 AA (ZIKV polyproteome position 14251445 of Zika Uganda Strain MR766 or position 1429-1449 of Zika Uganda Strain MR766-NIID).

The identified major epitope of the first study is only at the beginning, namely 13 amino acids, GDITWEKDAEVTG [SEQ ID NO. 2], part of an ordered 3D crystal structure, as shown in FIG. 4. The missing 8 amino acids of this epitope (highlighted in the circle) are part of a probably unordered linear protrusion of 54 amino acid length (dotted line), connected to an amino acid (highlighted with a star).

In a second more extended study, the inventors further characterized the IgG and IgM antibody profiles of Zika patients in a more comprehensive manner Therefore, they translated the proteomes of 19 Zika virus strains taken from sequencing data from Nicaragua, Colombia, Guatemala, Honduras, Mexico and Panama 100 additional Zika virus proteomes from the USA with a higher sequence heterogeneity into 4,356 different overlapping Zika virus peptides printed in duplicate on high-density peptide arrays for a high-resolution proteome-wide epitope screening.

The inventors used plasma of 100 Zika virus patients with a confirmed dengue virus naïve status in the acute and convalescent phase (200 samples), plasma of 50 Zika virus patients with a confirmed dengue virus positive status in the acute and convalescent phase (100 samples), 50 plasma samples of dengue virus patients in the convalescent phase taken before the Zika virus outbreak as disease control and plasma of 25 individuals with a confirmed dengue virus and Zika virus negative status as healthy controls. The samples were pre-collected and purchased from the Nicaraguan Biorepository at the Sustainable Sciences Institute and tested for Dengue (DENV) and Zika (ZIKV) viremia by polymerase chain reaction. The multiplexed IgG and IgM analysis was enabled by highly specific fluorescently labeled anti-human IgG and anti-human IgM secondary antibodies with different fluorescence wavelengths for emission and detection.

As with the first study, all Zika virus strains carry the major epitope in the polyproteome at various positions. The inventors also investigated the time-dependent antibody profiles in the Zika virus patients by comparison of samples from the acute vs. the convalescent stage. The proteome-wide microarray analyses showed specific IgG responses towards NS2b, especially in convalescent, but also in the acute samples (see FIG. 6, only protein NS2b shown). Almost no signals were observed in disease and healthy control samples. Interestingly, no specific IgM response towards NS2b was observed, neither in acute nor in convalescent samples.

The inventors could confirm the major epitope of the first study comprising the amino acid sequences WEKDAEVTGNSPRLD (SEQ ID NO. 9) or KDAEVTGNS (SEQ ID NO. 10).

In a second screening round of the second study, microarrays with peptide substitution scans of linear WT peptide WEKDAEVTGNSPRLD (SEQ ID NO. 9) and cyclic WT peptide KDAEVTGNS (SEQ ID NO. 10) were used to determine essential, conserved and less-conserved/variable amino acid positions and to identify stronger responding peptide variants. Each amino acid position in the WT sequence was systematically exchanged by all 20 physiologic amino acids, and the resulting linear and cyclic peptides and peptide variants were printed in triplicate on the peptide substitution scan microarrays. The immunoreactivity of the peptide variants was tested by incubation with Zika acute and convalescent plasma samples. The assays were carried out in a multiplexed format with fluorescently labeled secondary anti-human IgG and anti-human IgM antibodies to simultaneously analyze the IgG and IgM profiles of each sample.

The inventors identified as strongly conserved amino acid positions K7, E10, T12, G13, N14 in the IgG specific epitopes (wherein the numbering is in reference to SEQ ID NO. 1). In these positions, no amino acid exchange is tolerated. Other amino acid positions were less conserved or even variable. Strongest IgG reactivities were observed for peptide variants of A9P,E; V11D,E; S15D,K and R17D,E (see FIG. 7). Amino acid position K7 turned out to be essential for binding of anti-Zika virus antibodies in all tested human samples, and was shown to be differential for Zika virus in the corresponding regions of NS2b of other flaviviruses or chikungunya virus. More interestingly, not a single human sample exhibited any specific IgM reactivity against WT peptide WEKDAEVTGNSPRLD (SEQ ID NO. 9) or cyclic WT peptide KDAEVTGNS (SEQ ID NO. 10) and the corresponding peptide variants even in the acute phase (data not shown). The linear WT peptide WEKDAEVTGNSPRLD (SEQ ID NO. 9) and cyclic WT peptide KDAEVTGNS (SEQ ID NO. 10) and selected substituted peptide variants with strongest IgG reactivities were further evaluated by peptide ELISAs. The following examples and drawings illustrate the present invention without, however, limiting the same thereto.

### BRIEF DESCRIPTION OF THE DRAWINGS

FIG. 1: Course of disease and diagnostics (detection of RNA and antibodies).

A typical time course of virus and antibody kinetics in (flavi-)viral infections after virus infection: After infection, the virus replicates, until the immune system successfully overcomes the infection, generating specific IgM and eventually (after isotype switch) IgG antibodies against the viral proteins.

FIG. 2: First screening round (Arrays with whole proteome content):

(a) The IgG antibody reactivity of Zika patients of different groups was screened against the whole ZIKV proteome (only protein NS2b shown). The most prominent interaction was observed with the protein NS2b. A representative selection of data for relevant peptides (bracket with black asterisk) is shown in (b). Framed peptides represent Seq-IDs NO. 2-7. Fluorescence values are shown in gradual 3-color scale (green: >0; black: >100; red: >1000)

FIG. 3: Second screening round (Specific arrays with NS1, NS2a, NS2b content).

The IgG antibody reactivity showed clear signals in the NS2b protein. A major epitope was identified.

FIG. 4: 3D structure of the NS2b-NS3 serine protease complex.

The identified major epitope is only at the beginning (13 amino acids) part of an ordered 3D crystal structure (GDITWEKDAEVTG) [SEQ ID NO. 2], the missing 8 amino acids of this epitope (highlighted in the circle) are part of a probably unordered linear protrusion of 54 amino acids length (orange line), connected to the amino acid (highlighted with a star).

FIG. 5: Comparison of specific Zika NS2b and dengue NS2b reactivity in patients with and without dengue/Zika infection:

The identified major epitope in the Zika NS2b protein is only prevalent in patients after infection with Zika (convalescent samples, highlighted in circles). In the dengue virus NS2b protein, there is almost no reactivity.

FIG. 6: Comparison of specific Zika NS2b IgG reactivity in the second study in Zika and Dengue patients.

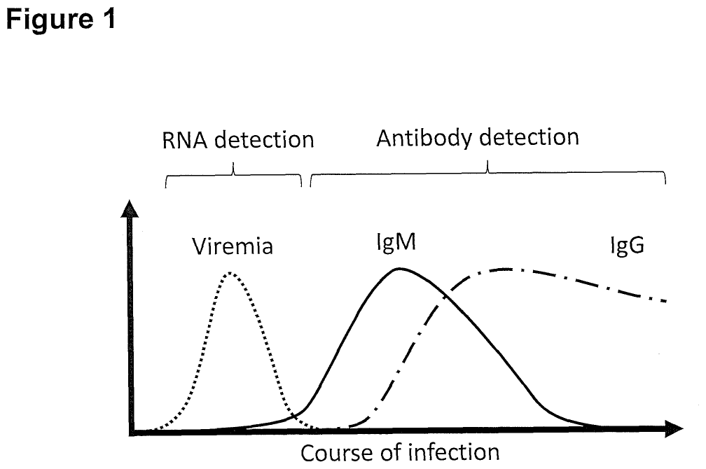

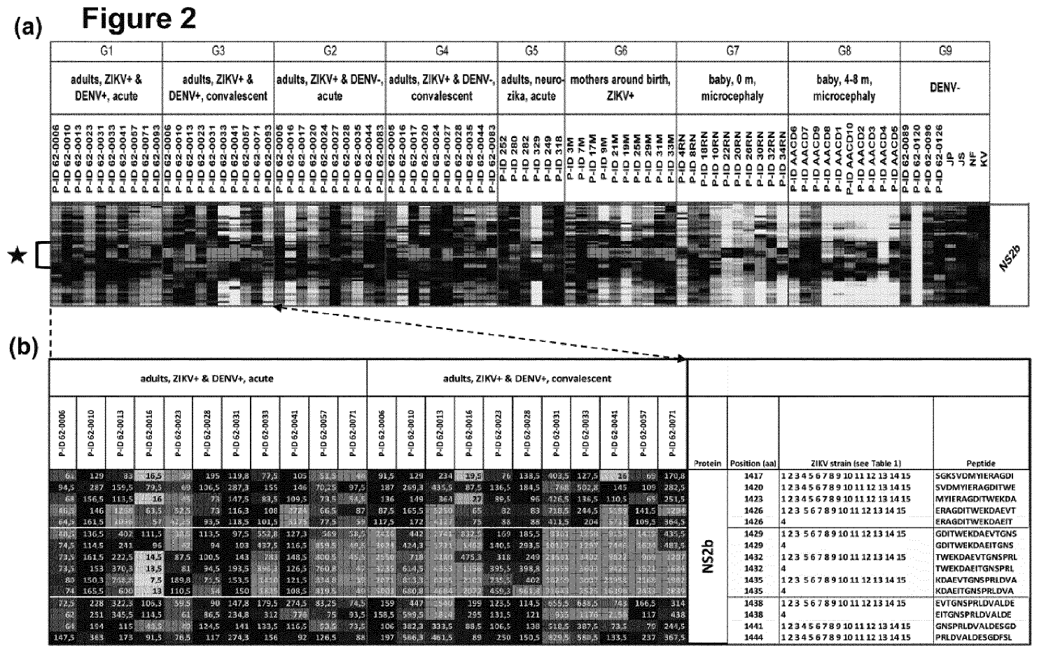

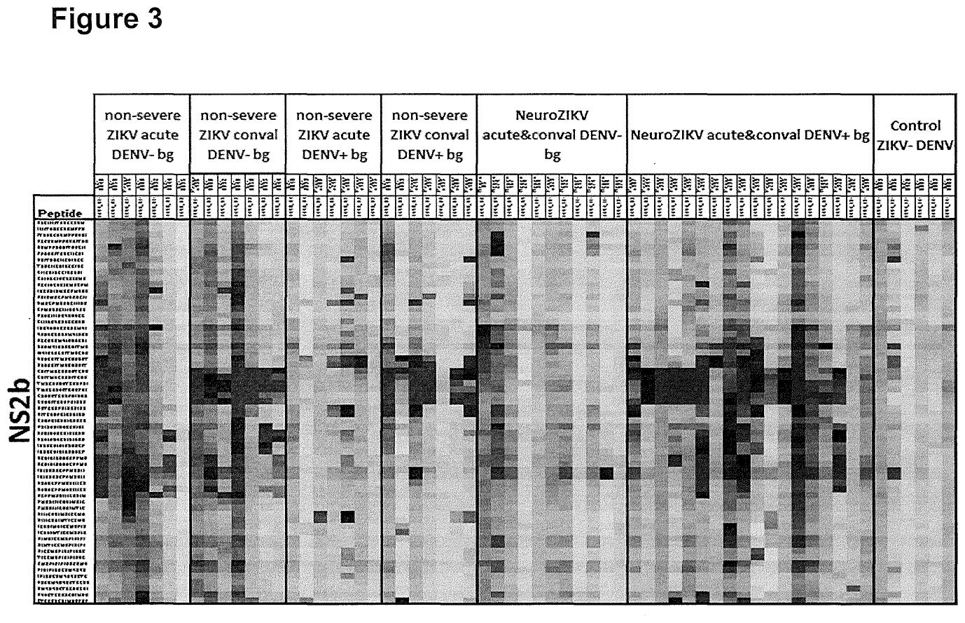

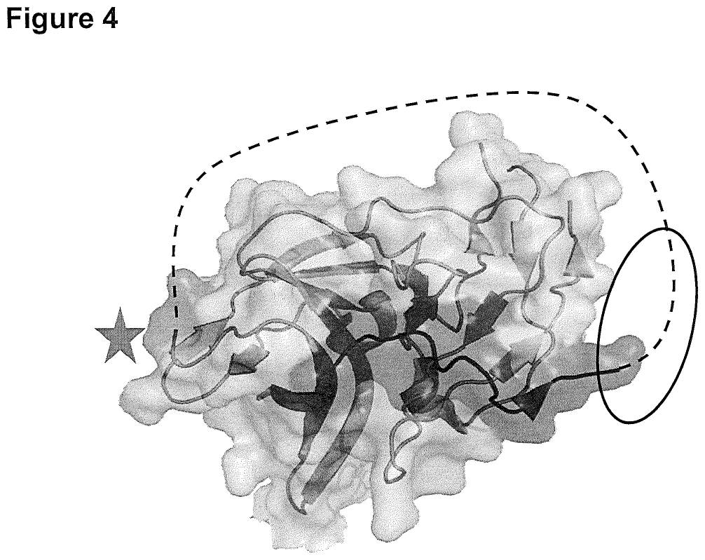

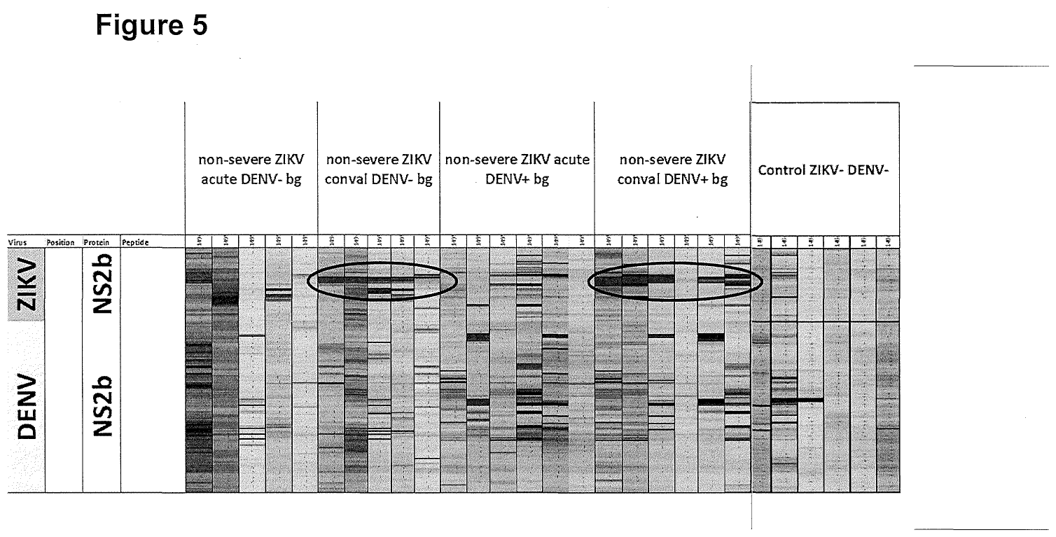

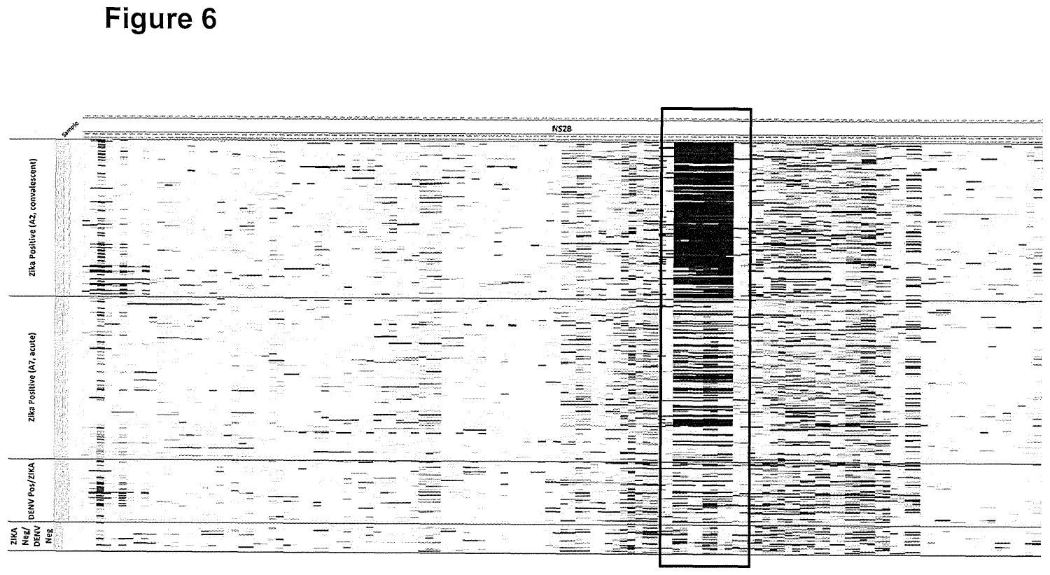

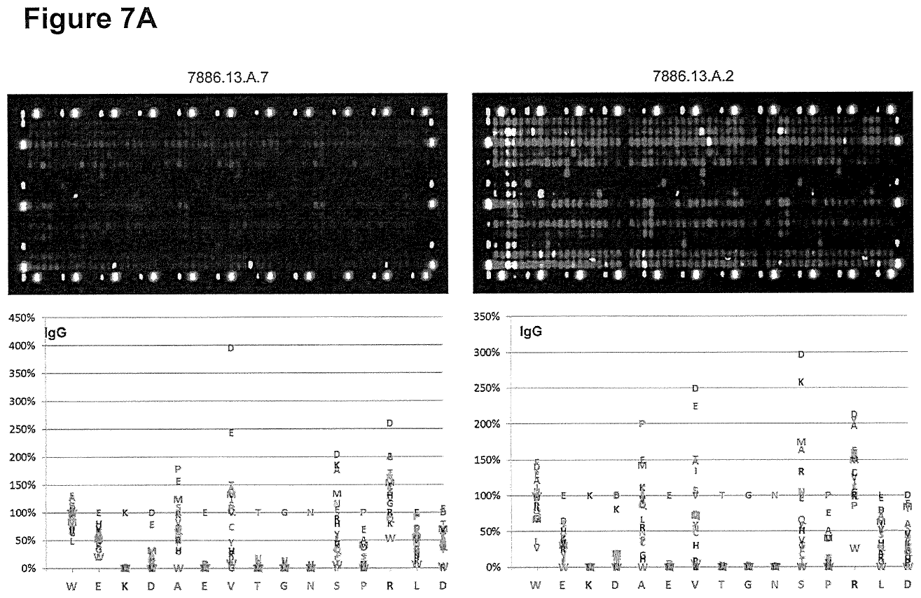

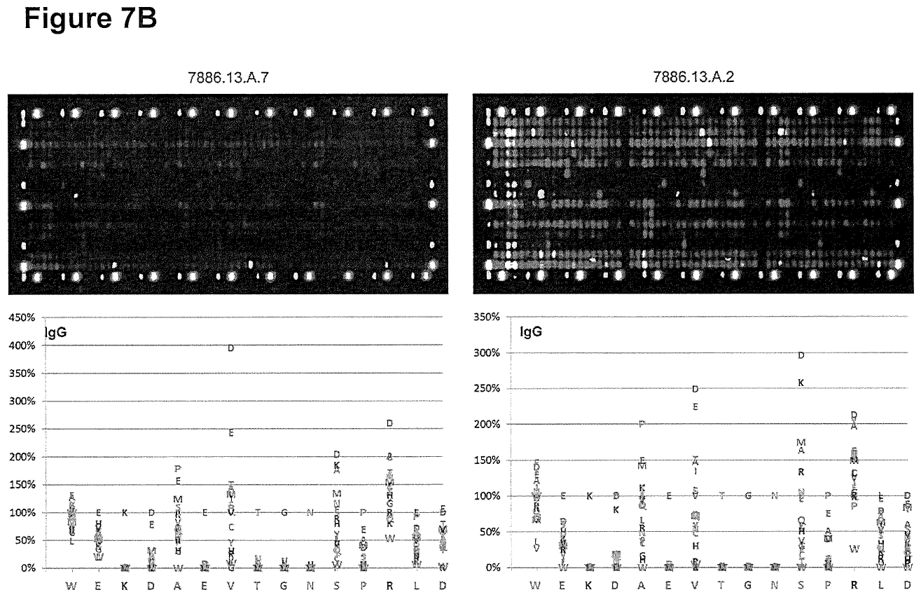

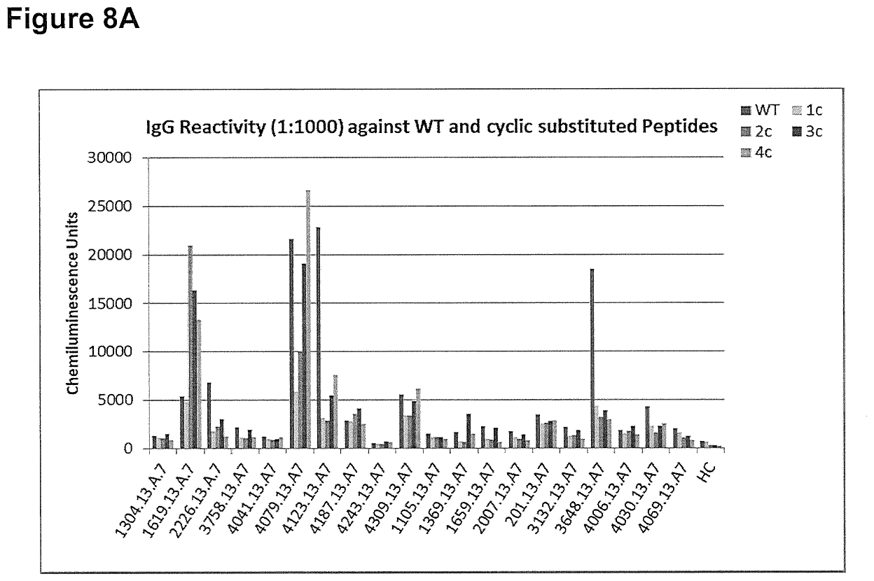

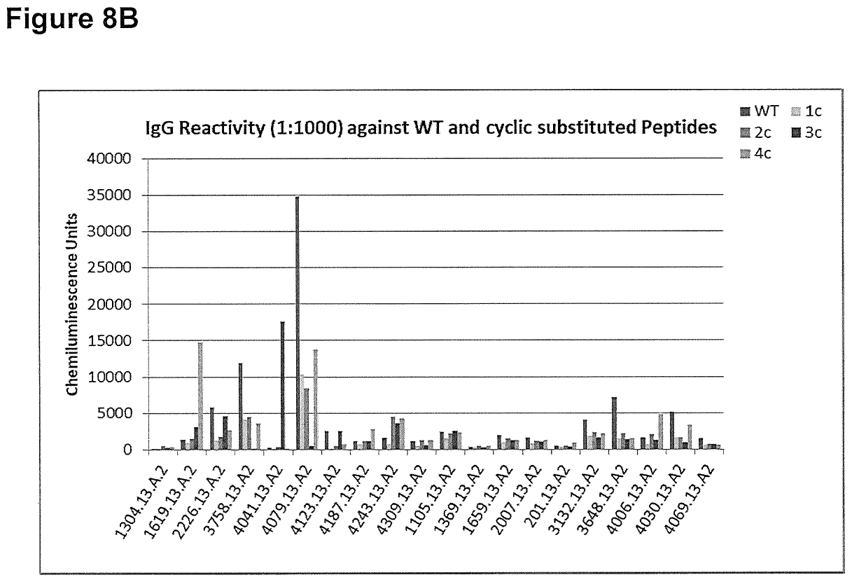

The second more comprehensive study analyzed the IgG and IgM antibody responses to the proteomes of 119 Zika virus strains using a total of 375 disease and control human plasma samples. While we did not observe any specific IgM reactivity against framed peptides representing SEQ ID NOs. 1-10, the major epitope in the Zika NS2b protein shows a clear and differential IgG reactivity in patients after infection with Zika in nearly all convalescent and a high number of acute samples. In contrast, the Zika negative/Dengue positive and Zika negative/Dengue negative samples hardly showed any IgG reactivity. Fluorescence values are shown in gradual 3-color scale (light gray: >0; gray: >100; dark gray: >1000)

FIG. 7: Full substitution scan of SEQ ID 9.

7A and B, top: Fluorescence pattern of four arrays stained with Zika acute and convalescent plasma samples. Arrays are framed with polio and hemagglutinin peptides as positive controls.

7A and B, bottom: Amino acid plots calculated from the IgG fluorescence intensities. The IgG reactivity of each amino acid exchange and each peptide variant was referenced to the IgG fluorescence intensity of wild type peptide WEKDAEVTGNSPRLD (SEQ ID NO. 9) set to 100%. The wild type peptide sequence is plotted from the N- to the C-terminus on the x-axis, the relative IgG reactivity of each amino acid substitution as percentage of the wild type peptide at 100% on the y-axis.

FIG. 8: IgG reactivity in different patient sera towards the WT peptide and cyclic Zika NS2b peptide variants.

The cyclic peptides and WT peptide were tested by a chemiluminescence ELISA using sera of Zika acute and Zika convalescent patients. A healthy control (HC) sera was included. The ELISA assays were performed under stringent conditions, that is at high serum dilutions (1:1000) to better evaluate the diagnostic potential of the peptide marker. The ELISA results are given in chemiluminescence units (CLUs). Samples were considered as positive, if the CLU of a respective sera is >=2 times over HC value. Each Zika serum exhibited a positive response towards at least one of the cyclic peptide variants.

A) IgG reactivity towards test peptides in Zika acute patient sera; the numbers at the y-axis indicate the chemiluminescence intensity, serum identifier are plotted at the x-axis.

B) IgG reactivity towards test peptides in Zika convalescent patient sera; the numbers at the y-axis indicate the chemiluminescence intensity, serum identifier are plotted at the x-axis.

### EXAMPLES

1. NS2b ZIKV Peptide Microarray

The whole proteome sequences of all at that time point publicly available 15 zika virus strains were retrieved from the NCBI database and translated into 15-mer peptides with a peptide-peptide overlap of 12 amino acids. Peptide arrays were produced by the company PEPperPRINT GmbH (Heidelberg, Germany) in a laser printing process on glass slides, coated with a PEGMA/PMMA graft copolymer, which were functionalized with a βAla-βAla-linker. In brief, a layer of amino acid particles, containing Fmoc-amino acid pentafluorophenyl esters, was printed layer after layer onto the functionalized glass slides, with intermittent melting (i.e. coupling) steps at 90° C. and chemical washing and capping steps (Stadler et al., 2008). Peptides were generated in duplicates on the arrays, which were screened for IgG responses in human sera.

2. Proteome-Wide Zika Virus Peptide Microarrays

The proteome sequences of 119 Zika virus strains from Nicaragua, Colombia, Guatemala, Honduras, Mexico, Panama and the USA were obtained from sequencing data. A homology analysis and sequence alignment were applied to remove redundant sequences, and unique Zika virus sequences translated into microarrays with 4356 different 15 amino acid peptides, which were printed in duplicate. Peptide microarrays were produced by PEPperPRINT GmbH (Heidelberg, Germany) in a laser printing process on glass slides, coated with a PEGMA/PMMA graft copolymer, which were functionalized with a βAla-βAla-linker. In brief, a layer of amino acid particles, containing Fmoc-amino acid pentafluorophenyl esters, was printed layer after layer onto the functionalized glass slides, with intermittent melting (i.e. coupling) steps at 90° C. and chemical washing and capping steps (Stadler et al., 2008).

3. Immunostaining of Peptide Microarrays

Peptide microarrays were placed in incubation trays (PEPperPRINT GmbH, Heidelberg, Geimany) and blocked for 30 min at room temperature at 120 RPM orbital shaking with western blot blocking buffer MB-070 (Rockland, USA).

In the first study, sera were diluted 1:1000 in PBS buffer with 0.05% Tween 20 pH 7.4 (PBST) and 10% blocking buffer, incubating the sera for 16 h at 4° C. and 50 RPM orbital shaking. Peptide microarrays were washed three times shortly with PBST, followed by an incubation with a 1:2500 dilution of the secondary antibody (goat anti-human IgG Fc specific DyLight 680, Rockland, USA), together with the control antibody, diluted 1:500 (anti-c-Myc antibody, PEPperPRINT, Germany), for 30 min at room temperature and 120 RPM orbital shaking. The peptide microarrays were washed 3×10 s with PBST and rinsed with deionized water. After drying in a stream of air, fluorescent images were acquired using an Odyssey Imaging System (LI-COR, USA) at 700 nm with a resolution of 21 μm and a scanning sensitivity of 7. Image analysis and quantification was performed with the PepSlide Analyzer software (Sicasys Software GmbH, Heidelberg, Germany).

In the second study, human plasma samples were diluted 1:250 in PBS buffer with 0.05% Tween 20 pH 7.4 (PBST) and 10% blocking buffer, incubating the sera for 16 h at 4° C. and 140 RPM orbital shaking. Peptide microarrays were washed three times shortly with PBST, followed by an incubation with a 1:5000 dilution of the secondary antibodies (goat anti-human IgG Fc specific DyLight680 and goat anti-human IgM μ chain specific DyLight800, Rockland, USA) for 30 min at room temperature and 140 RPM orbital shaking.

Samples used for the peptide array experiments of the first study were collected from individuals with acute febrile illnesses enrolled in a prospective cohort study from May 2015 to April 2016. The cohort was established in an urgent health care clinic in the Recife Metropolitan Region as part of the International Research Consortium on Dengue Risk Assessment, Management and Surveillance-IDAMS (www.idams.eu; Jaenisch et al., 2013; Jaenisch et al., 2016). The age of patients from whom sera were used for the assays varied from 9 to 57 years old, where 7 were females and 5 were males. Sample collection was performed in the first day of recruitment (Day 1—acute sample), which following the IDAMS protocol corresponds to the period within the first 72h of the febrile period, and on the convalescent phase (Day 10-30 after recruitment—convalescent sample). For molecular viral diagnosis, quantitative real-time PCR (qRT-PCR) for Dengue (DENV) and Zika (ZIKV) viruses was performed. Protocols were slightly modified from previously reported assays (Warrilow et al., 2002; Lances et al., 2012; Lanciotti et al., 2007). Positive controls were viruses extracted from cell culture, and the negative control was water. As for serology, samples were assayed for anti-DENV IgM and IgG and anti-ZIKV IgM, through ELISA. The Panbio® Dengue Capture ELISA was used for the anti-DENV IgM and IgG assays, following the manufacturer's protocol. The anti-ZIKV IgM ELISA protocol was that of the Centers for Diseases Control and Prevention (CDC) (MMWR 2016; Granger et al., 2017).

According to the assays results, each sample was classified as:
1) ZIKV Positive/DENV Naïve, if they were positive for ZIKV qRT-PCR and negative for DENV qRT-PCR in the acute phase and/or positive for anti-ZIKV IgM with titers >2 times those for anti-DENV IgM in the convalescent phase, and negative for anti-DENV IgG in the acute phase;
2) ZIKA Positive/DENV Exposed, if they were positive for ZIKV qRT-PCR and negative for DENV qRT-PCR in the acute phase and/or positive for anti-ZIKV IgM with titers >2 times those for anti-DENV IgM in the convalescent phase, and positive for anti-DENV IgG in the acute phase;
3) Naïve, if they were negative for DENV and ZIKV qRT-PCRs in the acute phase, and negative for anti-DENV and anti-ZIKV IgM and IgG in both acute and convalescent phases.

In the second study, human plasma samples were pre-collected and purchased from the Nicaraguan Biorepository at the Sustainable Sciences Institute and tested by PCR for Dengue (DENV) and Zika (ZIKV) viremia by polymerase chain reaction. The day of enrollment after onset of symptoms was in the range of 4-6 days for acute plasma samples. Collection day of convalescent sample after onset of symptoms was in the range of 14-21 days. The 375 samples were classified as:
1) ZIKV Positive/DENV Naïve, if they were positive for ZIKV PCR and negative for DENV PCR in the acute or convalescent phase;
2) ZIKA Positive/DENV Positive, if they were positive for ZIKV PCR and positive for DENV PCR in the acute or convalescent phase;
3) ZIKA Negative/DENV Positive, if they were positive for DENV PCR in the convalescent phase and collected before the Zika virus outbreak in 2015.
2) ZIKA Negative/DENV Negative, if they were negative for ZIKV PCR and negative for DENV PCR.

4. Full Substitution Scan

A substitution analysis was performed with the WT peptide WEKDAEVTGNSPRLD (SEQ ID No. 9) and cyclic peptide KDAEVTGNS (SEQ ID NO. 10) to determine essential, conserved and less-conserved/variable amino acid positions and to identify stronger reacting peptide variants.

Each amino acid position in the WT sequence was systematically exchanged by all 20 physiologic amino acids, and the resulting peptides and peptide variants were printed in triplicate on peptide microarrays. The immunoreactivity of the peptide variants was tested by incubation with Zika acute and convalescent plasma samples. The assay was carried out in a multiplexed format with fluorescently labeled secondary anti-human IgG and anti-human IgM antibodies to simultaneously analyze the IgG and IgM profiles of each sample.

Results are shown in FIGS. 7A and 7B. The IgG specific epitopes are strongly conserved in positions K7, E10, T12, G13, N14 (numbering in reference to SEQ ID NO. 1). In these positions, no amino acid exchange is tolerated. Other amino acid positions are less conserved or even variable. Strongest IgG reactivities were observed for peptide variants of A9P,E; V11D,E; S15D,K and R17D,E. Interestingly, amino acid position K7 turned out to be essential for binding of anti-Zika virus antibodies in all tested human samples, and was shown to be differential for Zika virus in the corresponding regions of NS2b of other flaviviruses.

5. Cyclic Peptides

For ELISA tests, four cyclic peptides with a Biotin tag preceding the C-terminal cysteine residue were synthesized. The non-cyclic WT peptide (“WT peptide”) was synthesized with a C-terminal Biotin tag at the C-terminus, the amino acid sequence was identical to SEQ ID NO. 9.

The cyclic peptides were cyclized via a thioether bond. Therefore, a C-terminal cysteine residue was added to the amino acid sequence of the peptide and the N-terminus carried a bromoacetyl group. The cyclization was via the thiol group, i.e. the cysteine side chain, and the bromacetylated N-terminus.

| | | SEQ |
|---|---|---|
| | | ID |
| Peptide | | NO. |
| WT | WEKDAEVTGNSPRLD | 9 |
| Peptide 1c (cyclized WT) | 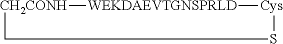 | 9 |
| Peptide 2c (cyclic, di-substituted) | 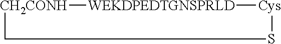 | 11 |
| Peptide 3c (cyclic, mono- substituted) | 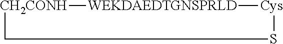 | 12 |
| Peptide 4c (cyclic, tri-substituted) | 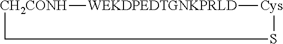 | 13 |

6. ELISA Tests

6.1 Protocol

Peptide ELISA-Tests were performed on Streptavidin functionalized 96 well microtiterplates (Lumitrac, Greiner BioOne, Germany). The plates were coated with the respective biotinylated peptides with a biotin tag positioned at the C-terminus, preceding the C-terminal cysteine. The plates were incubated overnight at 4° C. with test sera at a dilution of 1:1000 (v/v) in PBST/10% Rockland (PBST=Phosphate buffered saline pH 7.2, 0.05% Tween 20). Thereafter, plates were washed 3 times with PBST and incubated for 1 h at 20° C. with the detection antibody (goat anti-human Fc peroxidase conjugate) at a dilution of 1: 10000 in PBST/10% Rockland. The plates were washed again 3 times with PBST, followed by addition of the peroxidase substrate solution (BM Chemiluminescence ELISA substrate, Sigma Aldrich). The chemiluminescence was measured after 10 minutes at 425 nm using an automated plate reader (CLARIOstar®. BMG LABTECH GmbH, Germany). The chemiluminescence intensity is expressed in chemiluminescence units (CLU), the intensity expressing the immunoreactivity of tested sera.

6.2 Samples/Tested Sera

Samples used for the ELISA tests were pre-collected and purchased from the Nicaraguan Biorepository at the Sustainable Sciences Institute and tested for Dengue (DENV) and Zika (ZIKV) viremia by polymerase chain reaction.

6.3 ELISA Results:

The ELISA results are given in CLUs and shown in FIGS. 8A and 8B. Samples were considered as positive, if the CLU of a respective sera is >=2 times over HC value. All tested cyclic peptides and the WT peptide detected Zika specific antibody responses in acute patient sera as well as in convalescent patient sera. A positive IgG response was observed for each Zika serum to at least one of the tested peptide variants.

The features disclosed in the foregoing description, in the claims and/or in the accompanying drawings may, both separately and in any combination thereof, be material for realizing the invention in diverse forms thereof.

### REFERENCES

Brown, W. C.; Akey, D. L.; Konwerski, J. R.; Tarrasch, J. T.; Skiniotis, G.; Kuhn, R. J.; Smith, J. L., Extended surface for membrane association in Zika virus NS1 structure. Nature structural & molecular biology 2016, 23 (9), 865-7.
Chakraborty, S., MEPPitope: spatial, electrostatic and secondary structure perturbations in the post-fusion Dengue virus envelope protein highlights known epitopes and conserved residues in the Zika virus. F1000Research 2016, 5.
Dawes, B. E.; Smalley, C. A.; Tiner, B. L.; Beasley, D. W.; Milligan, G. N.; Reece, L. M.; Hombach, J.; Barrett, A. D., Research and development of Zika virus vaccines. npj Vaccines 2016, 1, 16007.
de Araújo, T. V.; Rodrigues, L. C.; de Alencar Ximenes, R. A.; de Barros Miranda-Filho, D.; Montarroyos, U. R.; de Melo, A. P.; Valongueiro, S.; de Albuquerque, M. F.; Souza, W. V.; Braga, C.; Filho, S. P.; Cordeiro, M. T.; Vazquez, E.; Di Cavalcanti Souza Cruz, D.; Henriques, C. M.; Bezerra, L. C.; da Silva Castanha, P. M.; Dhalia, R.; Marques-Júnior, E. T.; Martelli, C. M.; Group, i. f. t. M. E. R.; Health, B. M. o.; Organization, P. A. H.; Figueira, I. d. M. I. P. F.; Pernambuco, S. H. D. o., Association between Zika virus infection and microcephaly in Brazil, January to May, 2016: preliminary report of a case-control study. Lancet Infect Dis 2016, 16 (12), 1356-1363.
Granger D, Hilgart H, Misner L, Christensen J, Bistodeau S, Palm J, Strain A K, Konstantinovski M, Liu D, Tran A, Theel E S. Serologic Testing for Zika Virus: Comparison of three Zika Virus IgM-Screening Enzyme-Linked Immunosorbent Assays and Initial Laboratory Experiences. J Clin Microbiol 2017, 55 (7):2127-2136.
Jaenisch T, IDAMS, Sakuntabhai A, DENFREE, Wilder-Smith A, DengueTools: Dengue Research Funded by the European Commission—Scientific Strategies of Three European Dengue Research Consortia. PLOS Negl Trop Dis 2013, 7(12), e2320. (IF 2013 4, 489).
Jaenisch T, Tam D T H, Kieu N T T, Ngoc T V, Nam N T, Kinh N V, Yacoub S, Chanpheaktra N, Kumar V, Lum L C S, Sathar J, Pleites Sandoval E, Maron Alfaro G M, Laksono I S, Mahendradhata Y, Sarker M, Ahmed F, Caprara A, Benevides B S, Marques E, Magalhaes T, Brasil P, Netto M, Tami A, Bethencourt S E, Guzman M, Simmons C P, Quyen N T H, Merson L, Dung N T P, Beck D, Wirths M, Wolbers M, Lam P K, Rosenberger K, Wills B: Clinical evaluation of dengue and identification of risk factors for severe disease: protocol for a multicentre study in 8 countries. BMC Infectious Diseases 2016, 16:120; doi: 10.1186/s12879-016-1440-3.
Johnson, A. J.; Martin, D. A.; Karabatsos, N.; Roehrig, J. T., Detection of anti-arboviral immunoglobulin G by using a monoclonal antibody-based capture enzyme-linked immunosorbent assay. J Clin Microbiol 2000, 38 (5), 1827-31.
Johnson, B. W.; Kosoy, O.; Hunsperger, E.; Beltran, M.; Delorey, M.; Guirakhoo, F.; Monath, T., Evaluation of chimeric Japanese encephalitis and dengue viruses for use in diagnostic plaque reduction neutralization tests. Clin Vaccine Immunol 2009, 16 (7), 1052-9.
Kuno, G., Serodiagnosis of flaviviral infections and vaccinations in humans. Adv Virus Res 2003, 61, 3-65.
Kuno, G.; Chang, G. J., Full-length sequencing and genomic characterization of Bagaza, Kedougou, and Zika viruses. Arch. Virol 2007, 152 (4), 687-96.
Lanciotti R S, Kosoy O L, Laven J J, Velez J O, Lambert A J, Johnson A J, et al. Genetic and serologic properties of Zika virus associated with an epidemic, Yap State, Micronesia, 2007. Emerg Infect Dis. 2008; 14(8):1232-9. pmid:18680646
Lazear, H. M.; Diamond, M. S., Zika Virus: New Clinical Syndromes and Its Emergence in the Western Hemisphere. J Virol 2016, 90 (10), 4864-75.
Lessler, J.; Chaisson, L. H.; Kucirka, L. M.; Bi, Q.; Grantz, K.; Salje, H.; Carcelen, A. C.; Ott, C. T.; Sheffield, J. S.; Ferguson, N. M.; Cummings, D. A.; Metcalf, C. J.; Rodriguez-Barraquer, I., Assessing the global threat from Zika virus. Science 2016, 353 (6300), aaf8160.
Lover, A. A., Zika virus and microcephaly. Lancet Infect Dis 2016, 16 (12), 1331-1332.
Maeda, A.; Maeda, J., Review of diagnostic plaque reduction neutralization tests for flavivirus infection. Vet J 2013, 195 (1), 33-40.
Martin, D. A.; Muth, D. A.; Brown, T.; Johnson, A. J.; Karabatsos, N.; Roehrig, J. T., Standardization of immunoglobulin M capture enzyme-linked immunosorbent assays for routine diagnosis of arboviral infections. J Clin Microbiol 2000, 38 (5), 1823-6.
MMWR. 2016. Announcement: guidance for U. S. laboratory testing for Zika virus infection: implications for health care providers. MMWR Morb Mortal Wkly Rep 65:1304.
Muller, D. A.; Young, P. R., The flavivirus NS1 protein: molecular and structural biology, immunology, role in pathogenesis and application as a diagnostic biomarker. Antiviral Res 2013, 98 (2), 192-208.
Musso, D.; Gubler, D. J., Zika Virus. Clin Microbiol Rev 2016, 29 (3), 487-524.
Rances E, Ye Y H, Woolfit M, McGraw E A, O'Neill S L. The relative importance of innate immune priming in Wolbachia-mediated dengue interference. PLoS Pathog. 2012; 8(2):e1002548. pmid:22383881
Stadler, V.; Felgenhauer, T.; Beyer, M.; Fernandez, S.; Leibe, K.; Guttler, S.; Groning, M.; Konig, K.; Torralba, G.; Hausmann, M.; Lindenstruth, V.; Nesterov, A.; Block, I.; Pipkorn, R.; Poustka, A.; Bischoff, F. R.; Breitling, F., Combinatorial synthesis of peptide arrays with a laser printer. Angew. Chem. Int. Ed. Engl. 2008, 47 (37), 7132-5.
Stettler, K.; Beltramello, M.; Espinosa, D. A.; Graham, V.; Cassotta, A.; Bianchi, S.; Vanzetta, F.; Minola, A.; Jaconi, S.; Mele, F.; Foglierini, M.; Pedotti, M.; Simonelli, L.; Dowall, S.; Atkinson, B.; Percivalle, E.; Simmons, C. P.; Varani, L.; Blum, J.; Baldanti, F.; Cameroni, E.; Hewson, R.; Harris, E.; Lanzavecchia, A.; Sallusto, F.; Corti, D., Specificity, cross-reactivity, and function of antibodies elicited by Zika virus infection. Science 2016, 353 (6301), 823-6.
Waggoner, J. J.; Pinsky, B. A., Zika Virus: Diagnostics for an Emerging Pandemic Threat. J Clin Microbiol 2016, 54 (4), 860-7.
Warrilow D, Northill J A, Pyke A, Smith G A. Single rapid TaqMan fluorogenic probe based PCR assay that detects all four dengue serotypes. J Med Virol. 2002; 66(4):524-8. pmid: 11857532
Watterson, D.; Modhiran, N.; Young, P. R., The many faces of the flavivirus NS1 protein offer a multitude of options for inhibitor design. Antiviral Res 2016, 130, 7-18.
Zhang Z., Li Y., Loh Y. R., Phoo W. W., Hung A. W., Kang C., Luo D. Crystal structure of unlinked NS2B-NS3 protease from Zika virus. Science. 2016 doi: 10.1126/science.aai9309.

1-26. (canceled)

27. A protein NS2b or at least one fragment thereof as a biomarker or diagnostic marker for use in a method for the diagnosis and/or prognosis of Zika virus infections.

28. The NS2b protein or at least one fragment thereof according to claim 27, wherein the fragment comprises an amino acid sequence of the ZIKV polyproteome of positions 1429-1449 of Zika Uganda Strain MR766_NIID (SEQ ID NO: 1).

29. The NS2b fragment according to claim 27, wherein the fragment comprises or is a peptide having
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1.

30. The NS2b fragment according to claim 29, wherein the amino acid substitution(s) are
A9P, A9E, A9M, A9S, A9T, A9K;
V11D, V11E, V11T, V11A, V11N, V11S, V11M, V11L or I11D, I11E, I11T, I11A, I11N, I11 S, I11M, I11L;
S15D, S15K, S15M, S15A, S15R, S15N; and/or
R17D, R17E, R17T.

31. The NS2b fragment according to claim 27, wherein the fragment comprises or is a cyclic peptide having
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization.

32. A peptide or a cyclic peptide that is either:
A) a peptide comprising an amino acid sequence selected from SEQ ID NOs: 1 to 10 having one, two, three or four amino acid substitution(s) in position 9, 11, 15 and/or 17 in reference to the amino acid sequence of SEQ ID NO: 1 or
B) a cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization.

33. The peptide of claim 32 having a length of 5 to 130 amino acids.

34. The peptide or the cyclic peptide of claim 32, comprising at least one further component(s), which is/arc selected from
label(s),
tag(s),
linker or anchoring group(s)
or combinations thereof,
wherein said at least one further component(s) is/are covalently coupled to said peptide via a linker, an amino acid side chain, and/or to the N- and/or C-terminus.

35. A composition or an array comprising:
(a) at least one peptide comprising
an amino acid sequence selected from SEQ ID NOs: 1 to 10, or
an amino acid sequence having at least about 50% sequence identity to an amino acid sequence of SEQ ID NOs: 1 to 10, or
(b) at least one peptide comprising
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
and/or
(c) at least one cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization,
(d) a protein or at least one fragment thereof of claim 1.

36. A multimer compound comprising at least two of component(s) (a) to (d):
(a) a peptide comprising
an amino acid sequence selected from SEQ ID NOs: 1 to 10, or
an amino acid sequence having at least about 50% sequence identity to an amino acid sequence of SEQ ID NOs: 1 to 10, or
(b) a peptide comprising an amino acid sequence selected from SEQ ID NOs: 1 to 10 having one, two, three or four amino acid substitution(s) in position 9, 11, 15 and/or 17 in reference to the amino acid sequence of SEQ ID NO: 1,
(c) a cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization,
and/or
(d) a protein NS2b or at least one fragment thereof of claim 1,
wherein at least two of component(s) (a) to (d) are covalently coupled to each other or are connected via a linear or cyclic scaffold.

37. A peptide-based compound comprising:
(i) at least one peptide comprising
an amino acid sequence selected from SEQ ID NOs: 1 to 10, or
an amino acid sequence having at least about 50% sequence identity to an amino acid sequence of SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
and/or at least one cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1
wherein the cyclization is via
thioether formation, wherein the peptide comprises an additional cysteine residue at or near the C-terminus,
or
head-to-tail cyclization
and
(ii) at least one further component coupled to the peptide and/or cyclic peptide (i).

38. The peptide-based compound of claim 37, wherein the peptide and/or cyclic peptide has a length of at least 5 amino acids.

39. The peptide-based compound of claim 37, wherein the at least one further component is selected from
label(s),
tag(s),
linker or anchoring group(s),
or combinations thereof,
wherein the at least one further component(s) is/are preferably covalently coupled to said peptide and/or cyclic peptide via a linker, an amino acid side chain, and/or to the N- and/or C-terminus.

40. A method for the diagnosis and/or prognosis of Zika virus infections, comprising the steps of
(a) providing a sample of a patient to be tested,
(b) providing
(1) protein NS2b or at least one fragment thereof;
(2) a peptide-based compound comprising
(i) at least one peptide comprising
an amino acid sequence selected from SEQ ID NOs: 1 to 10, or
an amino acid sequence having at least about 50% sequence identity to an amino acid sequence of SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
and/or at least one cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1
wherein the cyclization is via
thioether formation, wherein the peptide comprises an additional cysteine residue at or near the C-terminus,
or
head-to-tail cyclization
and
(ii) at least one further component coupled to the peptide and/or cyclic peptide (i);
(3) a peptide comprising an amino acid sequence selected from SEQ ID NOs: 1 to 10 having one, two, three or four amino acid substitution(s) in position 9, 11, 15 and/or 17 in reference to the amino acid sequence of SEQ ID NO: 1;
(4) at least one cyclic peptide comprising
an amino acid sequence selected from SEQ ID NOs: 1 to 10, or an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization
(5) a composition or an array comprising
(a) at least one peptide comprising
an amino acid sequence selected from SEQ ID NOs: 1 to 10, or
an amino acid sequence having at least about 50% sequence identity to an amino acid sequence of SEQ ID NOs: 1 to 10, or
(b) at least one peptide comprising
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
and/or
(c) at least one cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization,
(d) a protein NS2b or at least one fragment thereof of claim 1;
and/or
(6) a multimer compound comprising at least two of component(s) (a) to (d):
(a) a peptide comprising
an amino acid sequence selected from SEQ ID NOs: 1 to 10, or
an amino acid sequence having at least about 50% sequence identity to an amino acid sequence of SEQ ID NOs: 1 to 10, or
(b) a peptide comprising an amino acid sequence selected from SEQ ID NOs: 1 to 10 having one, two, three or four amino acid substitution(s) in position 9, 11, 15 and/or 17 in reference to the amino acid sequence of SEQ ID NO: 1,
(c) a cyclic peptide comprising
an amino acid sequence selected from SEQ ID NOs: 1 to 10, or
an amino acid sequence selected from SEQ ID NOs: 1 to 10 having one, two, three or four amino acid substitution(s) in position 9, 11, 15 and/or 17 in reference to the amino acid sequence of SEQ ID NO: 1,
wherein the cyclization is via
thioether formation, wherein the peptide comprises an additional cysteine residue at or near the C-terminus,
or
head-to-tail cyclization,
and/or
(d) a protein NS2b or at least one fragment thereof,
wherein at least two of component(s) (a) to (d) are coupled to each other or are connected via a linear or cyclic scaffold;
(c) performing an immunoassay, comprising detecting an anti-Zika antibody response in said patient sample.

41. The method of claim 40, comprising the use of a negative control, wherein said negative control is a NS2b peptide, cyclic peptide or a peptide-based compound comprising an amino acid substitution in position K7 in reference to the amino acid sequence of SEQ ID NO: 1.

42. The method according to claim 40, wherein the NS2b fragment comprises an amino acid sequence of the ZIKV polyproteome of positions 1429-1449 of Zika Uganda Strain MR766 NIID (SEQ ID NO: 1)

43. The method according to claim 40, wherein the NS2b fragment has the following substitutions:
A9P, A9E, A9M, A9S, A9T, A9K;
V11D, V11E, V11T, V11A, V11N, V11S, V11M, V111, or I11D, I11E, MT, I11A, I11N, I11S, I11M, I11L;
S15D, S15K, S15M, S15A, S15R, S15N; and/or
R17D, R17E, R17T.

44. The method according to claim 40, wherein one fragment is used that comprises more than one of said peptides, or more than one fragment each comprising or consisting of one peptide are used.

45. The NS2b fragment according to claim 29, wherein the amino acid substitution(s) are A9P, A9E, V11D, MD, V11E, I11E, S15D, S15K, and/or R17D, R17E.

46. The NS2b fragment according to claim 29, comprising an amino acid sequence selected from SEQ ID NOs: 11 to 16.
